(19) United States
(12) Patent Application Publication
(10) Pub. No.: US 2022/0155134 A1
(43) Pub. Date: May 19, 2022

(54) Method and System to Track Weight

(71) Applicant: Daniel Lin, San Francisco, CA (US)

(72) Inventor: Daniel Lin, San Francisco, CA (US)

(21) Appl. No.: 17/666,474

(22) Filed: Feb. 7, 2022

Related U.S. Application Data
(63) Continuation-in-part of application No. 16/667,907, filed on Oct. 30, 2019, now Pat. No. 11,243,110.
Application No. 16/667,907 is a continuation-in-part of application No. 16/150,245, filed on Oct. 2, 2018, now Pat. No. 10,466,094.
Application No. 16/150,245 is a continuation-in-part of application No. 14/708,218, filed on May 9, 2015, now abandoned.
(60) Provisional application No. 61/991,327, filed on May 9, 2014.

(51) Int. Cl. G01G 19/44; A61B 5/00

(52) U.S. Cl. CPC G01G 19/44; A61B 5/6892; A61B 5/6891

(57) ABSTRACT

Embodiments herein provide systems and methods tracking the weight of an individual situated on a furnishing such as a chair or bed. One method generally includes receiving weight data at a weight sensor that is situated to recognize a change in weight on the furnishing, determining that a mobile device of the individual is proximately located to the weight sensor, and confirming by the mobile device that the change in weight is related to the individual.

### CROSS-REFERENCE TO RELATED APPLICATIONS

The present invention is a continuation-in-part of and claims the benefit of patent application Ser. No. 16/667,907 filed on Oct. 19, 2019, entitled “Method and System to Track Weight Without Stepping on a Weight Scale,” and issuing as Ser. No. 11,243,110 on Feb. 8, 2022, which is a continuation-in-part of and claims the benefit of patent application Ser. No. 16/150,245, filed on Oct. 2, 2018 and entitled “Method and System to Track Weight Without Stepping on a Weight Scale,” which is a continuation-in-part of and claims the benefit of patent application Ser. No. 14/708,218, filed on May 9, 2015 and entitled “Method and System to Track Weight Without Stepping on a Weight Scale,” which claims the benefit of U.S. provisional patent application 61/991,327 filed May 9, 2014 and entitled “Method and System to Track Weight Without Stepping on a Weight Scale,” the entirety of which are all hereby incorporated by reference.

### BACKGROUND

Modern day body weight scales not only measure a user's weight but also his body mass index (BMI), body fat, water weight, hydration percentage, heart rate, bone mass, air quality and various other health-related measurements (e.g., all such measurements generally referred to as “weight data” herein). These scales also store such weight data and enable an individual to track and analyze historical measurements. For example, the scales may include Bluetooth and/or Wi-Fi connectivity that enable them to communicate weight data to a corresponding software application (e.g., developed by a the scale's manufacturer or a partner thereof) running on the individual's computer system or mobile device, or to a corresponding service hosted by a third party (e.g., such as the scale's manufacturer) and accessible through the Internet (e.g., through a web application accessed through a browser) that stores the weight data, provides analytics on the weight data (e.g., to provide historical trends information and/or health advice to the individual), and provides a rich user interface for the user to interact with his weight data to gauge his overall health.

Despite the sophistication of these weight scales, many users do not use a weight scale consistently. Users typically experience some phases where they are more health conscious and are more likely to track their weight using a weight scale and other phases where they are less focused on health and are less likely to use the weight scale. Unfortunately, it is during the phases when users are less health conscious (e.g., and therefore are not tracking weight data) that weight and other health related attributes (that may be measured or indicated by weight data) may deteriorate and that the tracking of weight data would have otherwise provided an indication of such deterioration and/or encouragement to improve health.

Summary

Systems and methods for tracking the weight of an individual who may be situated on a furnishing such as an office chair or a bed are disclosed herein. By tracking the weight data for an individual while he is sitting on a chair (e.g., while working) or sleeping in bed, the individual does not need to consciously weigh himself every time he is interested in learning about his weight. Since the individual often goes to work each day (and sits in the chair) or sleeps in the same bed every night, his weight will be automatically measured without his conscious involvement.

In one embodiment, a weight sensor that is situated to recognize a change in weight on a furnishing (such as a bed or a chair) receives weight data, for example, as a result of the individual sitting or lying on the furnishing.

receiving weight data at a weight data sensor that is situated to recognize a change in weight on the furnishing;

confirming that a mobile device of the individual is proximately located to the weight data sensor; and

transmitting the received weight data to the mobile device of the individual,

confirming that the received weight data is related to the individual by comparing historic weight data of the individual available at the mobile device; and

transmitting the received weight data to a hosted service, wherein the hosted service provides a user interface to track weight patterns of the individual based on the received weight data.

The weight sensor confirms that a mobile device of the individual is proximately located to the weight sensor (e.g., by confirming a Bluetooth connection) and then the received weight data is transmitted to the individual's mobile device. The individual's mobile device (e.g., via an installed health application) is able to assess whether the received weight data is related to the individual by comparing it to historical weight data that is available on the mobile device (maintained on the mobile device or accessible through a service, etc.). If the mobile device confirms that the received weight data is related to the individual based on the historic weight data, then it transmits the received data to a hosted service that provides a user interface to track weight patterns of the individual.

In this manner, when the individual is interested in understanding his weight patterns, he need only log into the hosted service or view his mobile device to see his weight and does not need at that time to step on a weight scale, which may or may not be available at that time. Furthermore, the ability to silently track the individual's weight also enables the hosted service to provide alerts to the individual in the event of too much weight loss or gain during a phase where the individual may not be consciously weighing himself to track such loss or gain.

### BRIEF DESCRIPTION OF THE DRAWINGS

FIG. 1A depicts a weight data sensor that can be inserted underneath a mattress in accordance with one embodiment of the invention.

FIG. 1B depicts a weight data sensor that is embedded into a mattress in accordance with one embodiment of the invention.

FIG. 2 depicts a weight data sensor that is situated in an office chair in accordance with one embodiment of the invention.

FIG. 3 depicts a communication architectures for obtaining data from a weight data sensor in accordance with one embodiment of the invention.

FIG. 4 depicts a flow chart for obtaining data from a weight data sensor in accordance with one embodiment of the invention.

FIG. 5 depicts a user interface for an application that receives weight data in accordance with one embodiment of the invention.

### DETAILED DESCRIPTION

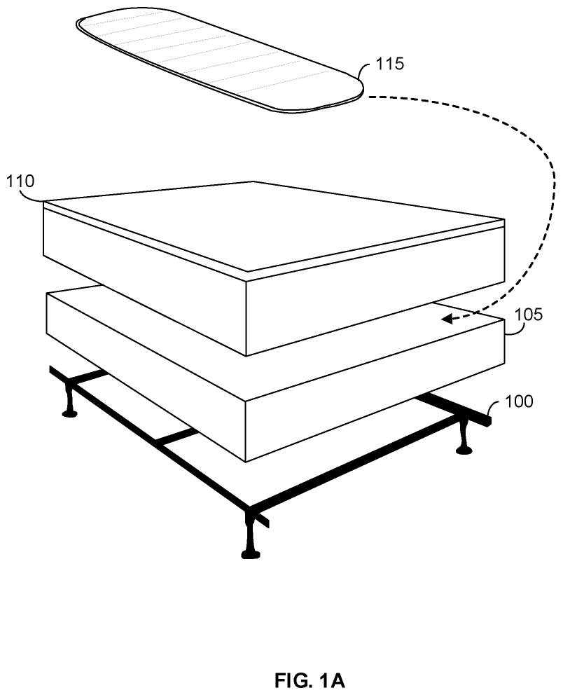

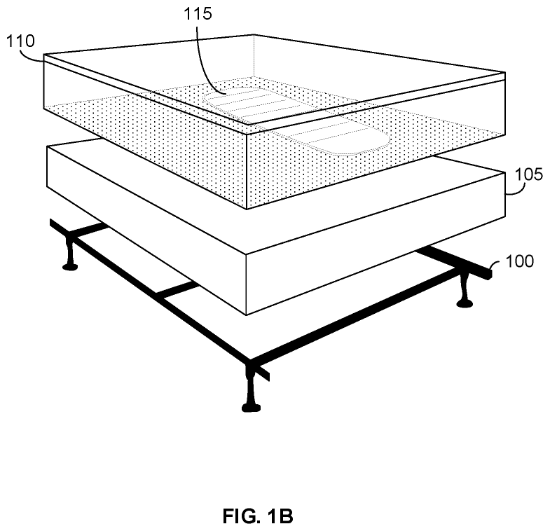

FIG. 1A depicts a weight data sensor that can be inserted underneath a mattress in accordance with one embodiment of the invention. A bed frame 100 supports a box spring 105 and a mattress 110. A weight data sensor 115 can be inserted underneath mattress 110, for example, between box spring 105 and mattress 110. As depicted in the embodiment of FIG. 1, weight data sensor 115 may take the form of an elongated pad that is long enough to experience an individual's full weight when the individual is lying on the bed, although it should be recognized that alternative form factors for weight data sensor 115 may be used consistent with the teachings herein. In addition to weight data, weight data sensor 115 may also be able to be able to monitor sleep patterns such as body movements, breathing cycles, heart rate, REM sleep patterns, deep sleep patterns, restless sleep patterns, etc.). Weight data sensor 115 may include Bluetooth, Wi-Fi or other radio wave or wireless communication capabilities that enable weight data sensor 115 to communicate with a local area network (LAN), a fitness device worn by an individual, a local computing device such as a smartphone, or with the Internet generally. In certain embodiments, weight data sensor 115 includes batteries or a power cord to plug into a nearby electric socket to provide power to drive the wireless communication capabilities and other digital functionality. Weight data sensor 115 also includes sensors to measure the weight (as well as other weight data in certain embodiments) of an individual when the individual is lying on mattress 110. For example, embodiments of a weight data sensor 115 may include a number of strain gauges (e.g., within a Wheatstone bridge, etc.) or other types of load cells (e.g., pneumatic, hydraulic, etc.) that measure compressive resistance change when an individual lies on mattress 110 and transmits a signal to a CPU or other circuit in weight data sensor 115 which convert the signal into a weight measurement. In certain embodiments, weight data sensor 115 may utilized air bladders to recognize change in pressure when an individual lies on the bed.

FIG. 1B depicts an alternate embodiment of weight data sensor 115 that is embedded into mattress 110 in accordance with one embodiment of the invention. In one embodiment, mattress 110 may be an adjustable air mattress that utilizes air chambers and the load cells of weight data sensor 115 are incorporated as part of the air chamber mechanism such that the load cells experience compressive resistance change (e.g., to measure an individual's weight) in response to changes in the air pressure of the air chamber (as opposed to a separate elongated pad or similar insert as depicted in FIG. 1B). It should be recognized that FIGS. 1A are 1B are merely examples for a use case of the invention when the furnishing is a bed and/or mattress and that there are a number of ways to design the form factor as well as the weight-measuring mechanisms of weight data sensor 115 in order to accurately measure an individual's weight when the individual is situated on any type of furnishing such as an office chair, lounge chair, dining chair or sofa, as well as mattress 110. For example, rather than a single weight data sensor 115 created as a insertion pad like FIG. 1A or embedded into a mattress like FIG. 1B, other embodiments may use 4 separate weight sensors that may be placed under each of the four feet of a bed frame. As is known in the art, these 4 weight sensors can be designed to coordinate their measurements to determine a weight changed experienced by the bed.

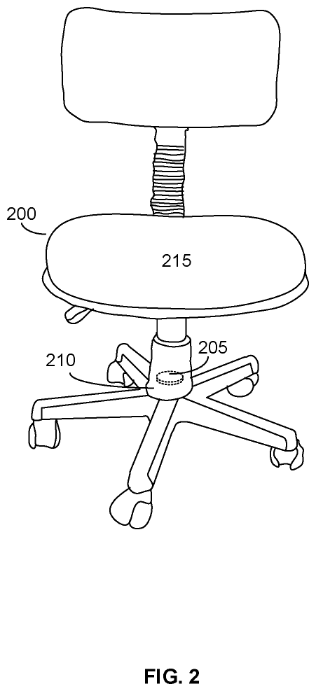

FIG. 2 depicts a weight data sensor 205 that is situated in an office chair 200 in accordance with one embodiment of the invention. Weight data sensor 205 is situated within the chair's cylinder 210 which bears the weight of the seat 215. Weight data sensor 205 can be functionality designed similarly to weight data sensor 115, for example, being configured with Bluetooth, Wi-Fi and/or other radio wave or wireless communication capabilities to communicate wirelessly with an individual's fitness device or smartphone or even to communicate with a hosted service as discussed further herein.

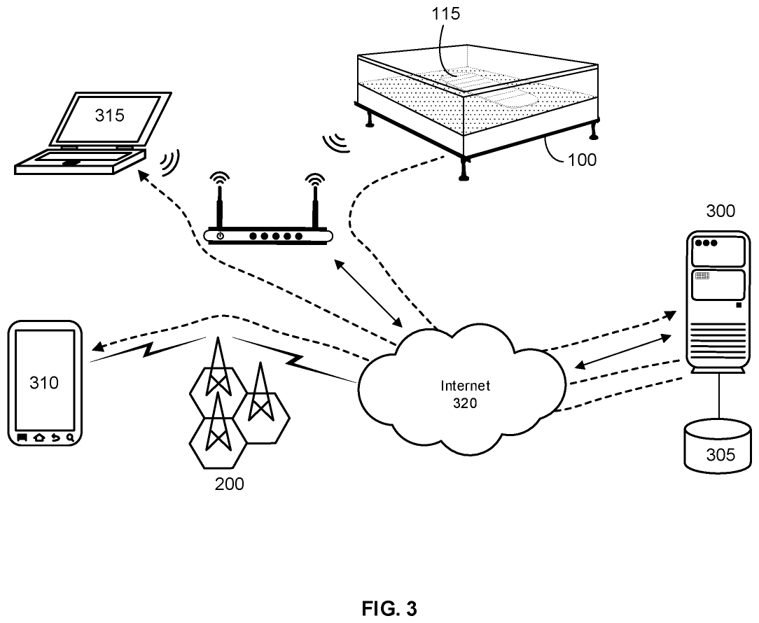

FIG. 3 depicts a communication architecture for obtaining data from a weight data sensor in accordance with one embodiment of the invention. In the embodiment of FIG. 3, weight data sensor (e.g., sensor 115 in bed frame 100 or sensor 25 in chair 200) wirelessly communicates over a LAN's Wi-Fi network through the Internet 320 to a hosted service 300 that stores weight data collected by weight data sensor 115 in database 305. Depending on embodiments, such communication may be direct or indirect, via a user's fitness device or health application on the individual's mobile device or smartphone. Hosted service 300 stores the weight data collected by the weight data sensor, performs analytics on the weight data and presents such weight data and analytics to the individual through a user interface, for example, through a web application accessible on a web browser (e.g., on laptop 310 or mobile device 315) or through an application installed on laptop 305 or mobile device 310. In an alternative embodiment, the weight data sensor may wirelessly communicate over a Bluetooth connection established with a user's laptop or mobile device (e.g., smartphone, tablet, etc.), which in turn, transmits weight data collected by the weight data sensor to hosted service 300 (e.g., through the LAN's Wi-Fl network and through the Internet). In certain embodiments, hosted service 300 may also receive other health-related data from other devices. For example, fitness bands or similar devices worn on the body of the individual may transmit heart rate, body temperature, ambient temperature, calories burned, steps walked, sleep patterns, location-based data and other data to hosted service 300 which can than combine such data with weight data collected from weight data sensor 115 and perform analytics thereon including as further discussed below. Such other health-related data is also stored in database 305, in addition to the weight data stored in database 305.

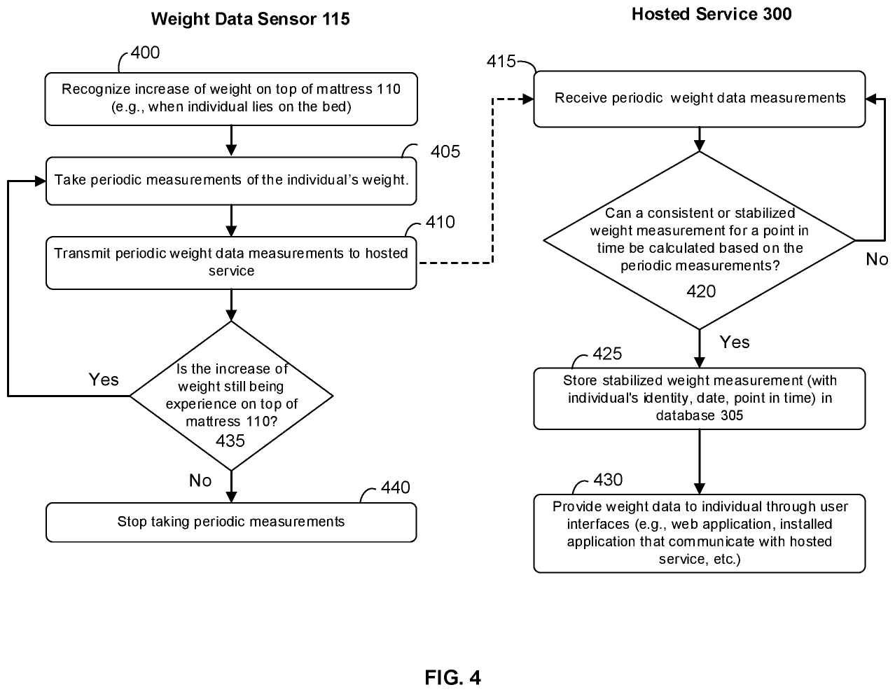

FIG. 4 depicts a flow chart for obtaining data from a weight data sensor in accordance with one embodiment of the invention. In step 400, the weight data sensor recognizes an increase of weight on top of a furnishing, such as mattress 110 or chair 200, due, for example, to an individual going to bed for the evening or sitting down to work. In step 405, the weight data sensor may take a measurement or begin to take periodic measurements (e.g., every 30 seconds, every minute, every 5 minutes, every 30 minutes, or any similar period depending on embodiment) of the weight of the individual in order to calculate an accurate measurement of the individual's weight based on multiple measurements. If the furnishing is a bed, for example the individual may need to fall asleep or otherwise stop moving on mattress 110 before the weight data sensor can determine or otherwise calculate a consistent and/or stabilized weight of the individual. If the furnishing is a chair, the weight data sensor may be able to take an accurate measurement more quickly. In step 410, the weight data sensor transmits the individual's weight measurements to hosted service 300, which receives them in step 415. Alternative embodiments may have the weight data sensor transmit its weight data to the individual's fitness band or smartphone, which in turn may either transmit the weight data to hosted service 300 or communicate amongst each other in order to ultimately communicate the weight data to hosted service 300. Furthermore, in certain embodiments, prior to step 410, the weight data sensor may confirm that either a fitness device of the individual or a smartphone of the individual (or a health application installed on the smartphone) is proximately located near the weigh data sensor (for example, through the confirmation of a Bluetooth connection between the weight data sensor and the fitness device and/or smartphone). Such a confirmation helps to increase the probability that the weight measurements related to the individual lying or siting on the bed or chair, rather than a different individual who is not in possession of the individual's fitness device or smartphone. In some embodiments if the fitness device or the smartphone are not confirmed to be proximate, the weight measurement is disregarded.

In the embodiment of FIG. 4, the transmitted weight measurements are the raw periodic measurements taken by weight data sensor 115. In step 420, hosted service 300 then performs heuristics and/or calculations to smooth the raw periodic measurements and determine whether a consistent and/or stabilized weight of the individual can be determined for a particular time based on the raw measurements. If, in step 420, a stable weight can be determined, then in step 425, hosted service 300 stores the stable weight measurement in database 305 in association with the individual's identity and the date (and possibly the particular time) and in step 430, provides such weight information to the individual through the user interface (e.g., web application, installed application on individual's device) as previously discussed. In certain embodiments, hosted service 300 is able to determine, based on comparisons with historic data (e.g., both weight data or other health-related data) stored in database 305, whether the determined weight is related or unrelated to the individual if, for example, multiple persons may sleep on mattress 110. In one example, if historic weight measurements are significantly different from the determined weight, hosted service 300 determines that the weight is unrelated to the individual. In certain embodiments, hosted service 300 may also receive other health-related data from other devices worn on the individual at generally the same time that weight measurements are received in step 415. If the received health-related data, such as a heart rate measurement from a fitness device worn by the individual, is significantly different than the historic measurements of the individual's heart rate while the individual is sleeping (as stored in database 305), then hosted service 300 could determine that the received weight measurement from the weight data sensor is unrelated to the individual (e.g., the individual is not sleeping in the bed, but rather actively awake elsewhere). And as previously discussed, in other embodiments, the weight data sensor may recognize when a fitness device that is worn by the individual is in close proximity to weight data sensor 115 (e.g., using proximity technologies such as Bluetooth or other similar radio communication technologies) and report such recognition to hosted service 300. If the fitness device is actively reporting other health-related data of the individual to hosted service 300 but is not in proximity to weight data sensor 115 when the determined weight is received in step 115, hosted service 300 may conclude that the determined weight is unrelated to the individual. In alternative embodiments, if the fitness device is not taking active measurements from the individual and is not in proximity to weight data sensor 115, weight data sensor 115 does not send any weight measurements to hosted service 305 in step 410. In yet other embodiments, weight data sensor 115 may recognize whether the individual's mobile device (such as a smart phone) is proximate to it before transmitting weight measurements to hosted service 300, or alternatively, transmit the weight measurements to the mobile device such that the mobile device can assess whether the received weight measurement are related to the individual. For example, a health-related application on the mobile device may maintain (or have access from hosted service 300) historic weight data that enables it to assess whether the received weight measurements are similarly enough the historic weight data. The mobile device may accept the received weight data if it concludes it related to the individual, and then send it to hosted service 300, or it may disregard such weight data if it concludes it is unrelated to the individual. Mobile devices of such embodiments may also receive, maintain or otherwise have access to other health related data from other fitness devices to further assist it in determining whether to accept or disregard the received weight data (as previously described). It should be understood that alternative embodiments may enable the mobile device of the individual to perform the steps depicted for hosted service 300 in FIG. 4, with weight data sensor 115 transmitting weight data to the individual's mobile device, which performs the analysis of whether to accept or disregard the weight data before transmitting it to hosted service 300.

Similarly, hosted service 300 may, in step 425, disregard raw data measurements that cannot be smoothed to generate a stable weight or are otherwise inconsistent with past measurements for the individual (e.g., a pet has jumped onto the bed, a guest is sleeping on the bed, etc.) In the embodiment of FIG. 4, in step 435, if weight data sensor 115 continues to experience the increased weight of the individual (e.g., while the individual is sleeping throughout the night), the flow will return to step 405 and continue to take measurements until the increased weight has been removed (or otherwise stop taking the measurements in step 440). It should be recognized that alternative flows for obtaining data from a weight data sensor in accordance with embodiments different from FIG. 4. For example, rather than having hosted service 300 perform the calculations on the raw periodic weight data measurement in step 420, in alternative embodiments, weight data sensor 115 may itself perform such calculations (and related smoothing techniques) and determine a stable weight for a particular point in time prior to transmitting the stable weight data to hosted service 100. Similarly, in alternative embodiments, weight data sensor 115 may transmit the weight data (either raw or stabilized) to a corresponding application running on a local device, such as laptop 310 or mobile device 315 (e.g., via Bluetooth, etc.) which in turn transmits the received data to hosted service 300.

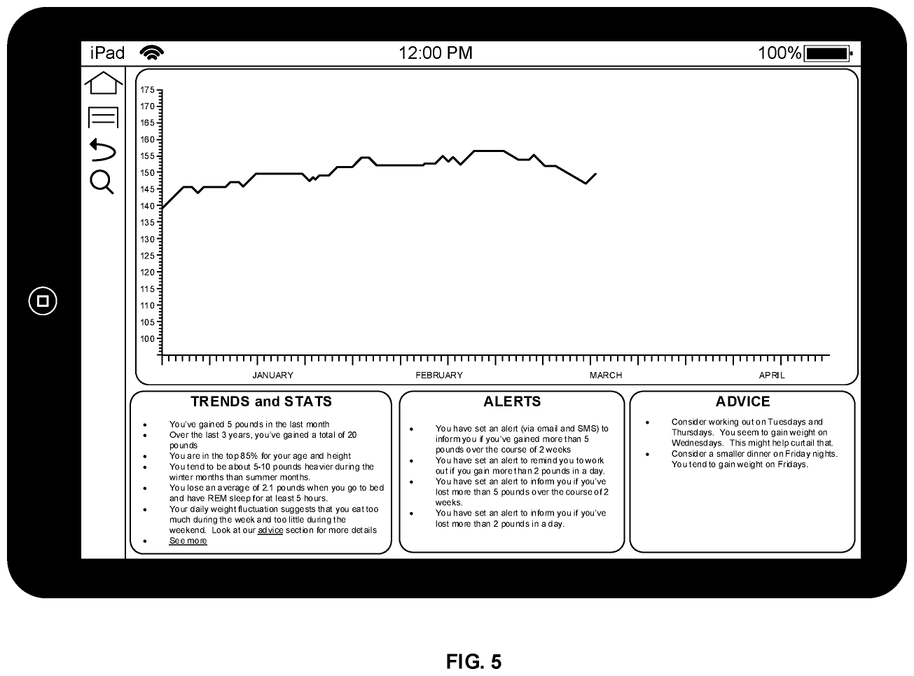

FIG. 5 depicts a user interface for an application that receives weight data in accordance with one embodiment of the invention. The user interface of FIG. 5 may be displayed in an application installed on a computing device such as a tablet, smartphone, laptop, desktop or other computer system that communicates with hosted service 300 or may be displayed in a web application running on hosted service 300 that is accessible through a web browser running on any of the foregoing. Hosted service 300 performs all the analytics and other computing needed to provide the information that the user interface of FIG. 5 displays to the individual. As depicted in FIG. 5, user interface 500 depicts a historic trends and statistics of weight measurements for an individual. While the granularity of the weight measurements is shown as a daily measurement, embodiments of user interface 500 may enable an individual to change the granularity (e.g., weekly, monthly, etc.), for example, to view trends at different granularities. User interface 500 also enables the individual to set alerts to notify the individual (e.g., via text message, email, etc.) if certain criteria related to weight data are recognized. For example, the individual may wish to be alerted if he gains more than 5 pounds over the course of a few weeks. User interface 500 may also identify trends regarding an individual's weight data. For example, user interface 500 has identified a trend that the individual tends to gain weight during the weekdays and also tends to be 5-10 pounds heavier during the winter months. User interface 500 may also provide comparisons of an individual's weight data against similarly situated individuals. For example, user interface 500 has identified that the individual is in the top 85% in weight for persons with similar height and age. In embodiments in which weight data sensor 115 also measures sleep patterns such as REM sleep patterns (or where host server 300 is able to combine such sleep pattern-related data from other devices such as fitness bands that transmit data to host server 300 as previously discussed), user interface 500 may also combine actual weight data with such other data to provide trends and/or statistics relating to a combination of the data. For example, as depicted in FIG. 5, hosted service 300 recognizes that the individual loses an average of 2.1 pounds when the individual has at least 5 hours of REM sleep in a night. The embodiment of FIG. 5 also provides advice to the individual regarding his weight depending upon the weight data trends and the statistics, for example, encouraging the individual to exercise on certain days or eat light meals on certain days given weight gain patterns. It should be recognized that the user interface of FIG. 5 is merely exemplary and many other user interfaces may be developed consistent with the teachings herein.

The various embodiments described herein may be practiced with other computer system configurations including hand-held devices, microprocessor systems, microprocessor-based or programmable consumer electronics, minicomputers, mainframe computers, and the like. One or more embodiments of the present invention may be implemented as one or more computer programs or as one or more computer program modules embodied in one or more computer readable media. The term computer readable medium refers to any data storage device that can store data which can thereafter be input to a computer system computer readable media may be based on any existing or subsequently developed technology for embodying computer programs in a manner that enables them to be read by a computer. Examples of a computer readable medium include a hard drive, network attached storage (NAS), read-only memory, random-access memory (e.g., a flash memory device), a CD (Compact Discs) CD-ROM, a CD-R, or a CD-RW, a DVD (Digital Versatile Disc), a magnetic tape, and other optical and non-optical data storage devices. The computer readable medium can also be distributed over a network coupled computer system so that the computer readable code is stored and executed in a distributed fashion.

Although one or more embodiments of the present invention have been described in some detail for clarity of understanding, it will be apparent that certain changes and modifications may be made within the scope of the claims. Accordingly, the described embodiments are to be considered as illustrative and not restrictive, and the scope of the claims is not to be limited to details given herein, but may be modified within the scope and equivalents of the claims. In the claims, elements and/or steps do not imply any particular order of operation, unless explicitly stated in the claims.

Plural instances may be provided for components, operations or structures described herein as a single instance. Finally, boundaries between various components, operations and data stores are only exemplary, and particular operations are illustrated in the context of specific illustrative configurations. For example, while step 410 notes that the weight data sensor transmits the individual's weight measurements to hosted service 300, alternative embodiments may have the weight data sensor sending the weight data to a fitness device or a smartphone and having the fitness device and/or smartphone further analyze the data and/or send the weight data to hosted service 300. Other allocations of functionality are envisioned and may fall within the scope of the invention(s). In general, structures and functionality presented as separate components in exemplary configurations may be implemented as a combined structure or component. Similarly, structures and functionality presented as a single component may be implemented as separate components. These and other variations, modifications, additions, and improvements may fall within the scope of the appended claims(s).

I claim:

1. A method for tracking the weight of an individual who may be situated on a furnishing, the method comprising the steps of:
receiving weight data at a weight data sensor that is situated to recognize a change in weight on the furnishing;
determining that a mobile device of the individual is proximately located to the weight data sensor; and
transmitting the received weight data to the mobile device of the individual,
confirming at the mobile device that the received weight data is related to the individual by comparing historic weight data of the individual available at the mobile device; and
transmitting the received weight data to a hosted service, wherein the hosted service provides a user interface to track weight patterns of the individual based on the received weight data.
